(19) United States
(12) Patent Application Publication
(10) Pub. No.: US 2022/0176321 A1
(43) Pub. Date: Jun. 9, 2022

(54) System and Process for Hybrid Membrane Distillation-Pervaporation

(71) Applicant: New Jersey Institute of Technology, Newark, NJ (US)

(72) Inventors: Somenath Mitra, Bridgewater, NJ (US); Sagar Roy, Newark, NJ (US); Oindrila Gupta, Harrison, NJ (US)

(73) Assignee: New Jersey Institute of Technology, Newark, NJ (US)

(21) Appl. No.: 17/543,488

(22) Filed: Dec. 6, 2021

Related U.S. Application Data
(60) Provisional application No. 63/123,198, filed on Dec. 9, 2020.

(51) Int. Cl. B01D 61/58; B01D 61/36; B01D 69/14; B01D 71/02; B01D 71/38; B01D 69/02; C02F 1/44; B01D 71/36; B01D 17/02; C07C 7/04

(52) U.S. Cl. CPC B01D 61/58; C02F 2101/34; B01D 61/362; B01D 61/366; B01D 69/148; B01D 71/021; B01D 71/38; B01D 69/02; C02F 1/447; C02F 1/448; B01D 71/36; B01D 17/02; C07C 7/04; B01D 2325/023; B01D 2311/2615; C02F 2209/40; B01D 61/364

(57) ABSTRACT

A membrane distillation (MD) system consisting of a membrane module and carbon nanotube immobilized membrane for organic solvent separation is disclosed. The MD module includes a feed inlet and outlet, a sweep gas inlet, and a sweep gas outlet. Thermostats are positioned at the feed inlet and outlet to measure the change in temperature. Preferential sorption of the organic on carbon nanotube immobilized membrane contributes to enhanced solvent removal of the MD system. A pervaporation (PV) system consisting of a membrane module and polyvinyl alcohol (PVA) mixed matrix membranes with graphene oxide (GO)—carbon nanotubes (CNTs) for enhanced purification of the alcohol solution after membrane distillation to remove trace amount of water is disclosed.

### CROSS-REFERENCE TO RELATED APPLICATION

The present application claims priority benefit to a U.S. provisional application entitled “System and Process for Hybrid Membrane Distillation-Pervaporation,” which was filed on Dec. 9, 2020, and assigned Ser. No. 63/123,198. The entire content of the foregoing provisional application is incorporated herein by reference.

### STATEMENT REGARDING FEDERALLY SPONSORED RESEARCH

This invention was made with government support under Agreement No. CBET-1603314 awarded by the National Science Foundation. The government has certain rights in the invention.

### FIELD OF THE DISCLOSURE

The present disclosure relates to biofuel and solvent recovery and/or concentration via a combined membrane distillation (MD)—pervaporation (PV) system. In particular, the present disclosure relates to bio-fuel recovery employing membrane distillation and sweep gas membrane distillation (SGMD).

### BACKGROUND

Ethanol forms an azeotrope with water once it reaches 89.4 mol % at 78° C. and atmospheric pressure. This mixture is hard to separate using a normal distillation process and can generally only be done through azeotropic distillation. Both azeotropic and conventional distillation consume substantial energy. The energy consumption for conventional distillation and azeotropic distillation are 10,376 and 3,305 kJ/kg ethanol, respectively. Apart from high energy consumption for azeotropic distillation, benzene, a highly carcinogenic and toxic substance used as an azeotropic dehydrating agent in many plants, is a major health concern. There are several proposals for ethanol removal, such as solvent extraction, gas stripping, for example, with CO₂ and vacuum evaporator flash tank, although great attention has also been directed toward membrane processes for this application. There are essentially two different membrane technology processes described in scientific literature for ethanol removal: membrane distillation (MD) and pervaporation (PV).

Membrane distillation (MD) is an emerging membrane separation technology that has a huge potential application in the fields of organic solvent recovery, desalination and water purification, wastewater treatment, fruit juice concentration, removal and recovery of low boiling components from aqueous mixtures, membrane crystallization, etc. During membrane distillation, a membrane serves as a barrier, allowing passage of water while retaining algae and/or other substances to be collected. In a conventional MD process, feed (e.g., saline water) flows on one side of a hydrophobic membrane and a distillate (e.g., cold water) flows on an opposite side of the hydrophobic membrane.

The feed solution is at an elevated temperature (e.g., 40° C.˜70° C.) in a membrane distillation process. This heated feed solution is transported into a membrane module, where vapor is generated from the temperatue difference and transported across the porous membrane. The highly hydrophobic membrane allows the vapor to pass through it, while repelling the liquid mixture from entering the membrane pores. A cool inert sweep gas sweeps the vapor coming through the membrane pores due to the presence of a vapor pressure gradient between the hot feed side and the cold permeate side.

The different MD configurations generally used to maintain a vapor pressure difference across a MD membrane include direct contact MD (DCMD), sweep gap MD (SGMD), air gap MD (AGMD), and vacuum MD (VMD). Sweep gas MD modules using carbon nanotube membranes have been demonstrated. A sweep gas MD module has the advantage of relatively low conductive heat loss and less energy consumption than vacuum MD.

Carbon nanotube (CNT) based membranes have been used in a variety of separation applications that range from pervaporation and extraction to nanofiltration. The physicochemical interaction between the solutes and the membrane can be dramatically altered by immobilizing CNTs on the membrane surface. First, CNTs are excellent sorbents that have surface areas between 100 and 1,000 m²/g. Many factors, such as the presence of defects, capillary forces in nanotubes, and polarizability of graphene structure, lead to strong sorbate/sorbent interactions, and the absence of a porous structure lead to high specific capacity while facilitating fast desorption of large molecules. A more recent development in MD has been the development of a carbon nanotube immobilized membrane (CNIM) for desalination where the CNTs increase the partitioning of the water vapor while rejecting hydrogen bonded salt-water phase leading to dramatic increase in flux.

Microwave irradiation incorporated into a SGMD process was demonstrated to reduce the concentration polarization and temperature polarization. Enhanced performance of SGMD with microwave irradiation was reported for organic solvent recovery for its use as biofuel(s). The solvent flux was significantly higher at an optimized feed temperature and concentration using microwave heating and carbon nanotube membranes. In these applications, a microwave oven was used for heating the feed stream before it entered the SGMD module.

In SGMD with CNTs, significant enhancement in solvent flux was attributed to the preferential sorption and fast desorption to the permeate side via CNTs serving as nanosorbents.

During conventional heating, the entire volume of the feed stream is uniformly heated. On the contrary, microwave heating involves direct heating of the feed mixtures resulting in localized superheating. The dielectric loss of solvents (in this case ethanol, and acetone, butanol, ethanol (ABE)) is known to increase with temperature whereas for water it decreases with temperature. The localized super heating and breaking of hydrogen bonded solvent-water clusters are bound to enhance the tendency of solvent molecules to escape from the feed mixture resulting in improved flux and better separation efficiency.

In pervaporation, liquid mixtures are separated by selective interaction of compounds with a dense membrane. The components are selectively transported through the membrane and then vaporized due to lower partial pressure in the permeate side, achieved using a vacuum pump or an inert gas stream. The separation is accomplished by relative permeation of solution through the membrane, which depends on both thermodynamic (adsorption) and kinetic (diffusion) aspects. MD is similar to the pervaporation process. The main difference between the two processes is the role of the membrane. The hydrophobic microporous membrane acts only as a support to the liquid-vapor interface and does not chemically distinguish solution components. The process selectivity depends on the vapor-liquid equilibrium phase separation.

Despite efforts to date, there is a continuing need to improve the recovery of biofuels and solvents and to reduce energy consumption associated therewith. These and other needs are satisfied by the systems and processes/methods disclosed herein.

### SUMMARY

In accordance with embodiments of the present disclosure, hybrid systems and processes/methods combining membrane distillation and pervaporation for ethanol separation and ABE separation from water are disclosed. Experimental data and data reported in the literature for PV were taken into account, carefully selected in terms of temperature and ethanol concentration. Experiments of MD and pervaporation were performed under the same operational conditions, put together in series. The model calculations of the present disclosure show that with 10 (v/v %) of ethanol in feed, a concentration of 73 (v/v %) for ethanol was achieved with MD. The permeate collected was then used as the feed composition for PV experiments. The PV experiments concentrated the ethanol solution up-to 96.4% with a recovery of 90% and removal of the excess water.

Methods and materials for sweep gas membrane distillation with microwave irradiation (MIMD) are described herein, where various embodiments of the materials and methods may include some or all of the elements and features described below. The materials and methods disclosed herein advantageously maximize vapor flux, thereby providing an enhanced solvent removal rate from the feed solution. Even though the current subject matter has specific application in alcohol concentration for use in industries and as biofuels, the materials and methods disclosed herein may be beneficially employed in other applications including, but not limited to, biofuels or solvent usage in paint or pharmaceutical industries.

Embodiments discussed herein include novel MD-PV systems with wide-ranging utility and applicability. Specifically, although generally described with reference to organic solvent removal/recovery for biofuels, the present disclosure is not limited to organic solvent removal/recovery for biofuels and may be used for a range of applications including, e.g., desalination and wastewater treatment.

In accordance with one or more embodiments, MD systems discussed herein generally include at least one SGMD module and at least microwave unit, and PV systems discussed herein generally include at least one PV membrane module and a liquid nitrogen trap to condense the permeated component.

In one or more embodiments, an MD module disclosed herein includes a feed inlet to receive an aqueous feed solution and a feed outlet, a condensing medium (sweep gas) inlet and outlet to obtain a condensing medium and to remove a stream of solvent vapor from the MD module, respectively.

In one or more embodiments, a PV module disclosed herein includes a MD feed inlet to receive an alcohol solution containing trace amount of water and a feed outlet, a condensing medium (vacuum) inlet and outlet to obtain a condensing permeate (liquid nitrogen trap) and to recirculate a stream of solvent from the PV module, collected as the retentate.

The MIMD system in sweep gas mode disclosed herein generally includes a microwave oven positioned to receive a feed stream before entering the SGMD membrane module. The microwave oven may have at least one liquid outlet connected to the feed inlet of the SGMD membrane module for one-pass operation. The SGMD membrane module is connected back to the feed reservoir to allow recirculation of the feed solution.

The membrane module may be employed in the form of a hollow fiber membrane module, a flat membrane module, or a spiral wound membrane module in one embodiment of the SGMD systems and methods disclosed herein.

In another embodiment, methods are disclosed for measuring the unknown concentration of the recirculated feed solution using UV-Vis spectroscopy and gas chromatography (GC) using a standard calibration curve. In one embodiment, a method is disclosed for downstream recovery of fermentation products using the MD-PV system.

In one or more embodiments, the MIMD and PV system disclosed herein includes flowmeters to measure the feed flow rate and sweep gas/vacuum flowrate connected to the feed and permeate inlet, respectively. The flowmeter functions to limit sweep gas/vacuum flow into the permeate channel to allow higher degree of air-sweeping in the permeate channel and thus enable higher degree of evaporation rate from the membrane module.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed hybrid membrane distillation and pervaporation system and method, and associated systems and methods, reference is made to the accompanying figures, wherein:

FIG. 1 is a schematic diagram of a MD-PV hybrid system in accordance with one or more embodiments of the present disclosure;

FIG. 2 is a schematic diagram of an experimental system used for solvent separation using membrane distillation in combination with a PV system in series in accordance with one or more embodiments of the present disclosure;

FIG. 3 is a schematic diagram of an experimental system used for solvent separation using membrane distillation in accordance with one or more embodiments of the present disclosure;

FIG. 4 is a schematic diagram of an experimental system used for solvent purification using pervaporation in accordance with one or more embodiments of the present disclosure;

FIGS. 5A-5C are SEM images of the surfaces of a plain PTFE membrane (FIG. 5A), a CNIM (FIG. 5B) and a CNIM-ODA construct (FIG. 5C) in accordance with one or more embodiments of the present disclosure;

FIG. 6A is a graphical depiction of TGA analysis of a CNIM & CNIM-ODA in accordance with one or more embodiments of the present disclosure and an unmodified PTFE membrane;

FIG. 6B is a graphical depiction of DSC curves of a CNIM & CNIM-ODA in accordance with one or more embodiments of the present disclosure and an unmodified PTFE membrane;

FIGS. 7A & 7B provide photograph of contact angle of 10% ethanol-water mixture on unmodified PTFE membrane (FIG. 7A), and CNIM (FIG. 7B), in accordance with one or more embodiments of the present disclosure;

FIGS. 8A-8C provide photographs of contact angle of ABE mixture (0.6, 1.2, and 0.4 v/v % of ABE) in unmodified PTFE membrane (FIG. 8A), CNIM (FIG. 8B), and CNIM-ODA (FIG. 8C), in accordance with one or more embodiments of the present disclosure;

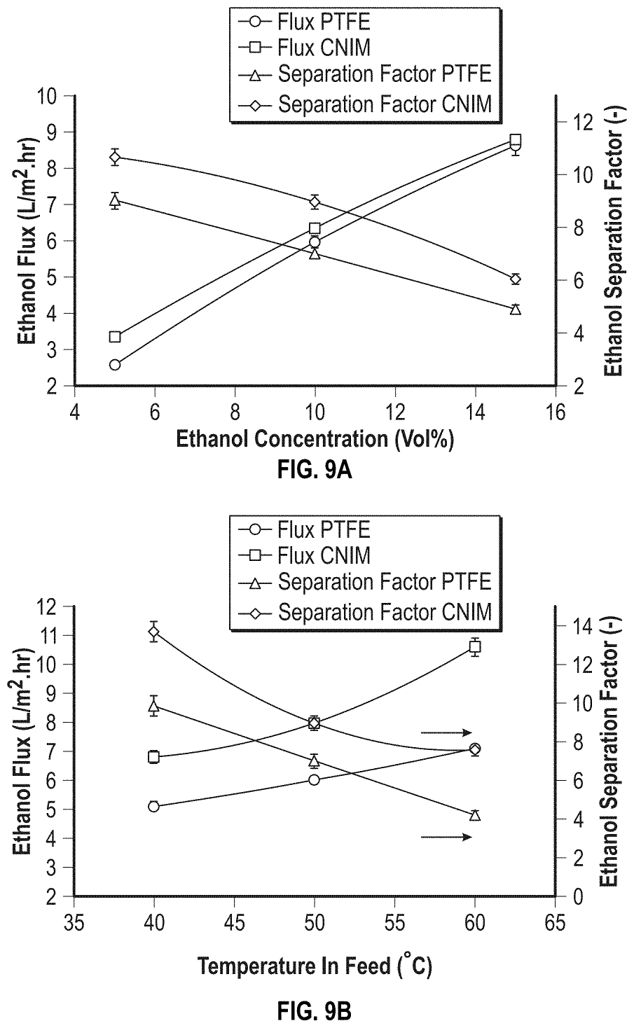

FIG. 9A illustrates graphical depictions of data reflecting ethanol flux and separation factor with CNIM and PTFE membrane as a function of the ethanol feed concentration at a feed flowrate of 112 mL/min and sweep gas flowrate of 4.5 L/min in accordance with one or more embodiments of the present disclosure;

FIG. 9B shows graphical depictions of data reflecting ethanol flux and separation factor with CNIM and PTFE membrane as a function of the feed temperature at a feed flowrate of 112 mL/min and sweep gas flowrate of 4.5 L/min in accordance with one or more embodiments of the present disclosure;

FIGS. 10A-10F are graphical depictions of data reflecting acetone (FIG. 10A), butanol (FIG. 10B) and ethanol (FIG. 10C) flux and separation factor (FIGS. 10D-F) with CNIM, CNIM-ODA and PTFE membrane as a function of the ABE feed concentration at a feed flowrate of 112 mL/min and sweep gas flowrate of 4.5 L/min in accordance with one or more embodiments of the present disclosure;

FIGS. 11A-11F are graphical depictions of data reflecting acetone (FIG. 11A), butanol (FIG. 11B) and ethanol (FIG. 11C) flux and separation factor (FIGS. 11D-F) with CNIM, CNIM-ODA and PTFE membrane as a function of the feed temperature at a feed flowrate of 112 mL/min and sweep gas flowrate of 4.5 L/min in accordance with one or more embodiments of the present disclosure;

FIG. 12 is a graphical depictions of data reflecting water flux and separation factor with PVA and PVA-GO-CNT membrane as a function of the feed water content at a feed flowrate of 20 mL/min and room temperature in accordance with one or more embodiments of the present disclosure;

FIG. 13 is a graphical depiction of microwave heating performance in terms of ethanol vapor flux and separation factor;

FIG. 14 is a graphical depiction of CNIM & CNIM-ODA performance in terms of ABE vapor flux and separation factor; and,

FIG. 15 is a graphical depiction of PVA-GO performance in terms of solvent vapor flux and separation factor.

### DETAILED DESCRIPTION

The present disclosure will now be described more fully hereinafter with reference to the accompanying drawings, in which illustrative embodiments of the present invention are shown. In the drawings, the relative sizes of regions or features may be exaggerated for clarity. The present disclosure may, however, be embodied in many different forms and should not be construed as limited to the embodiments set forth herein; rather, these embodiments are provided so that this disclosure will be thorough and complete, and will fully convey the scope of the invention to those skilled in the art. Well-known functions or constructions may not be described in detail for brevity and/or clarity.

The terminology used herein is to describe particular embodiments only and is not intended to limit the present invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

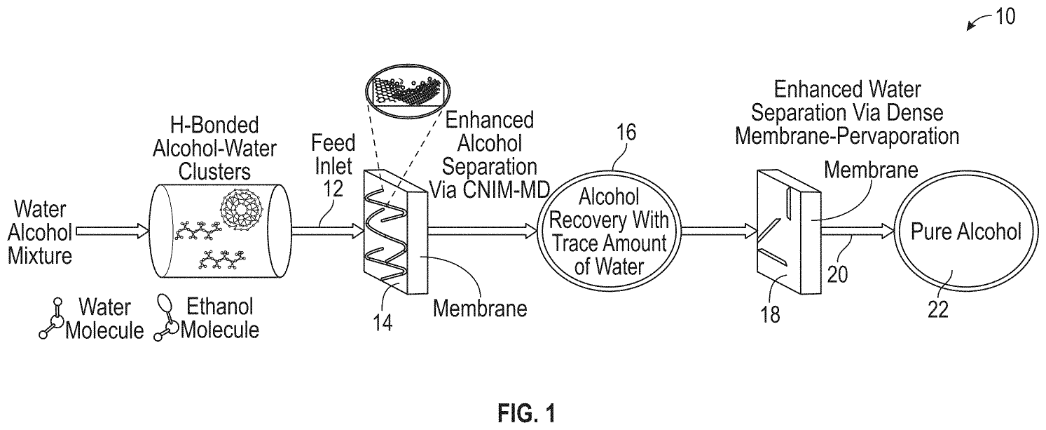

Now referring to FIG. 1, a flow chart is schematically depicted in which a water alcohol mixture forms H-bonded alcohol-water clusters that are fed to a CNIM-MD membrane that enables alcohol recovery with a trace amount of water. Enhanced water separation is achieved downstream by way of a dense membrane-pervaporation unit. Based on overall operation of the disclosed system, pure/substantially pure alcohol is recovered.

More specifically, in the exemplary embodiment of FIG. 1, a MD system 10 is provided wherein a feed stream (which includes H-bonded alcohol-water clusters) 12 is fed to a CNIM-MD membrane 14 that includes a polymeric membrane, which includes a layer including carbon nanotubes (CNTs) immobilized on a polytetrafluorethylene surface (PTFE). It will be understood that other suitable polymers besides PTFE could be employed. For the sake of brevity, the layer including CNTs immobilized in the PTFE may be referred to herein as the CNIM layer. The CNIM layer may be further disposed on a porous substrate. Also, in this embodiment, the alcohol recovery with trace amounts of water 16 is fed to a PV system 18 that includes a polymeric dense membrane, which includes a polyvinyl alcohol (PVA) base followed by graphene oxide (GO) to form a mixed matrix membrane (MMM). It will be understood that other bases could be employed. The effluent 20 from the PV system 18 constitutes pure/substantially pure alcohol 22.

The carbon nanotubes may be any suitable carbon nanotube, such as those commercially available from Cheap Tubes Inc., Brattleboro, Vt. The CNTs may be single or multi-walled. The diameter of the CNTs may range from about 1 nm to about 100 nm. The length of the CNTs may range from about 1 to about 25 μm. In some embodiments, the CNTs are amine functionalized.

In general, carbon nanotubes (CNTs) are excellent sorbents that have the ability to absorb organic solvents and desorb large molecules. As is known to those skilled in the art, many factors, such as the presence of defects, capillary forces in nanotubes, and/or polarizability of graphene structure, lead to strong sorbate/sorbent interactions. As used herein, in some embodiments, preferential sorption and fast desorption of the organic solvent to the permeate side via CNTs serving as nanosorbents is undertaken. The organophilic CNT surface is selective towards organic solvents due to its organic nature.

In accordance with certain embodiments, methods of making carbon nanotube-immobilized membranes may include the steps of dispersing a plurality of carbon nanotubes in acetone to form a carbon nanotube dispersion, dissolving a super-absorbent polymer in water to form a super-absorbent copolymer solution, adding the super-absorbent copolymer solution to the carbon nanotube dispersion to form a super-absorbent polymer-carbon nanotube mixture, applying the super-absorbent polymer-carbon nanotube mixture to a surface of a porous substrate and drying the super-absorbent polymer-carbon nanotube mixture. In some embodiments, the method may include adding octadecyl amine (ODA) to at least one of the plurality of carbon nanotubes prior to forming the dispersion.

Polyvinyl alcohol with high molecular weight may be obtained from Alfa Aesar and any suitable GO solution can be used, such as those commercially available from Sigma-Aldrich, St. Louis, Mo. Glutaraldehyde (grade II, 25 wt %) and hydrochloric acid (HCl) were acquired from Sigma-Aldrich and used without further purification.

### EXAMPLES & EXPERIMENTS

The materials and the methods of the present disclosure used in embodiments will be described below. While the embodiments disclose the use of specific compounds and materials, it is understood that the present disclosure could employ other suitable compounds or materials. Similar quantities or measurements may be substituted without altering the method embodied below.

Acetone (AR≥99.5%), butanol (anhydrous, 99.8%), and ethanol (anhydrous, ≥99.5%) were obtained from Sigma-Aldrich (St. Louis, Mo.). Deionized water (Barnstead 5023, Dubuque, Iowa) was used in the experiments and examples. Raw multi-walled carbon nanotubes (CNTs) were purchased from Cheap Tubes Inc., Brattleboro, Vt. The average diameters of the CNTs were about 30 nm and a length of up to 15 μm. Octadecyl amide (—CO—NH—C₁₈H₃₇) functionalization (CNT-ODA) was performed in the laboratory following a method described elsewhere[1]. The membrane employed for this MD experiment was a PTFE membrane on PP support (Advantec MFS, Inc.; Dublin, Calif., 0.2 μm pore size, 74% porosity).

1.5 mg of amine functionalized and raw CNTs were dispersed in acetone (10 g) via sonication for 3 hrs. The ODA functional groups provided good dispersibility. PVDF (0.2 mg) was added to the above solution, which acts as a binder.

PVA/GO MMMs were prepared by dense-film casting method and solvent evaporation. PVA powder (1.5 g) was dissolved under stirring in 50 mL of distilled water at 80° C. The obtained solution was filtered to remove any insoluble impurities. GO was added to the PVA solution to produce the dope suspension that was stirred during 12 h and processed by sonication. Afterwards, the in situ cross-linking procedure was performed by adding 0.1 mL of GA and 0.1 mL of HCl to the dope. This was stirred during 15 min, cast on a clean glass plate and then dried in an oven at 60° C. for 24 hours. Finally, the MMMs were peeled off of the glass plate.

The CNIM, CNIM-ODA, and unmodified PTFE membranes were characterized by using scanning electron microscopy (JEOL; model JSM-7900F). This was done by cutting the membranes into 0.5 cm long pieces and coating with carbon films. Thermogravimetric analysis (TGA) was used to investigate the degradation of modified membrane materials during heating. TGA was carried out using a Perkin-Elmer Pyris 7 TGA system at a heating rate of 10° C./min under air. Contact angle measurements were made to study the hydrophobic nature of the CNIM and CNIM-ODA membranes. These measurements were performed using a digital video camera mounted at the top of the stage.

The morphological structure of the membrane surface and cross-section of the cross-linked-PVA and its MMMs were evaluated using a field emission scanning electron microscope (JEOL; model JSM-7900F). The samples were coated through a sputtering process with gold-palladium (Au/Pd). The corresponding images were captured at suitable magnification.

Differential scanning calorimetry (DSC) was conducted on a 10 mg sample using a Mettler Toledo DSC822e system. The T_{g} routine was performed in two cycles from room temperature up to 450° C. at the temperature ramping of 20° C.·min⁻¹.

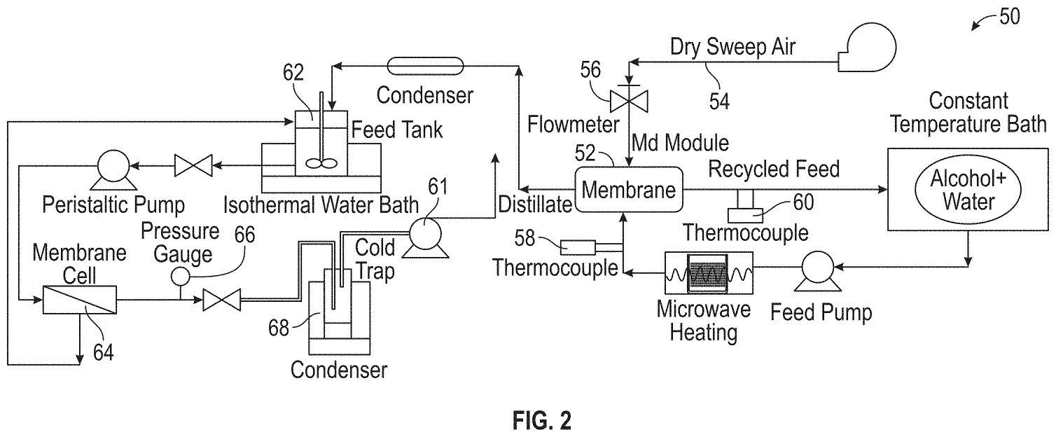

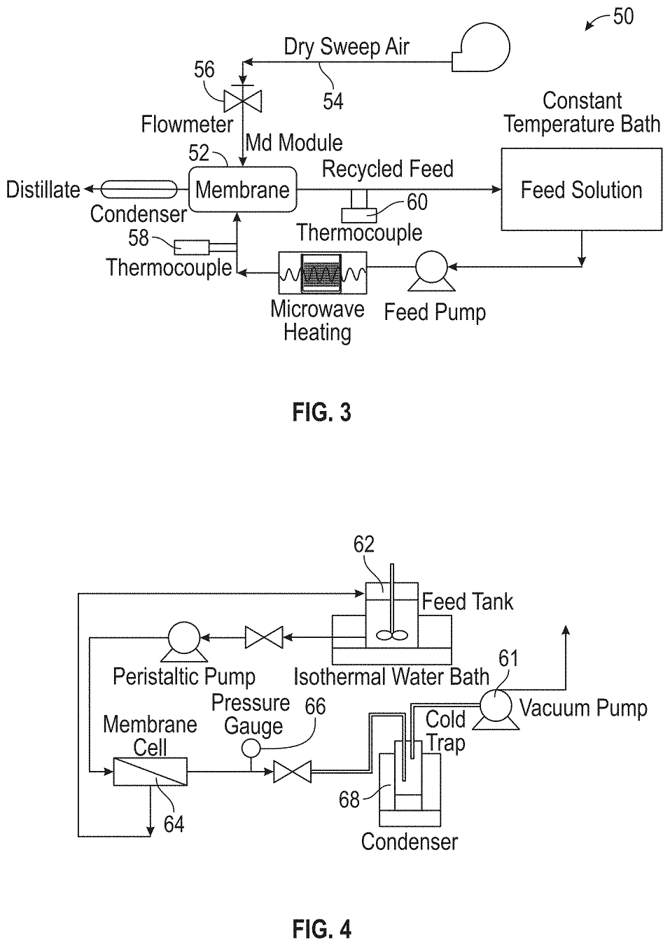

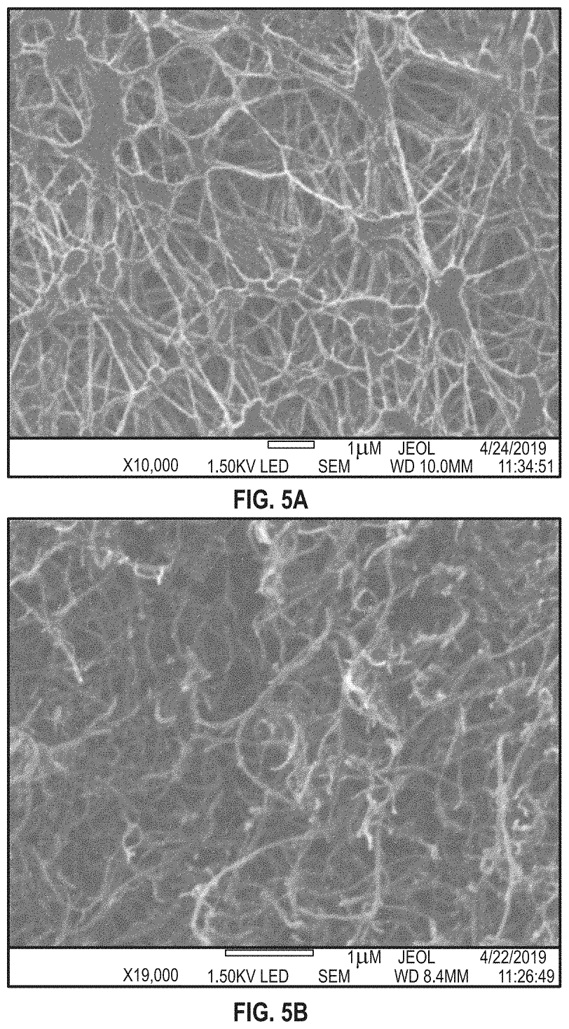

Now referring to FIGS. 2-4 (and consistent with the flow chart of FIG. 1), an experimental setup 50 for ethanol and ABE vapor removal from a simulated air stream is shown using MD and a combination of a MD-PV system. A flat membrane module 52 was used to make the SGMD test cell that was fabricated from polytetrafluoroethylene (PTFE). The desired solvent vapor concentration was achieved by carefully adjusting the flow rates of the sweep gas streams 54 using a flow controller, a flow meter, and a pressure gauge 56. The feed temperature were varied from 30-50° C. using thermistor thermometers 58, 60 (K-type, Cole Parmer) placed on the inlet and outlet of the stream. The ethanol solvent concentrations were varied 5-15 vol % and ABE concentration was varied in the ratio 3:6:1. A vacuum pump 61 was connected to the feed-side of the module and the feed was recirculated to measure the change in volume after each experiment. The highly hydrophobic membranes allowed solvent vapor to pass through the membrane preferentially. Laboratory air supplied was passed through the permeate side of the membrane 52 from the fume hood at room temperature (22° C.). To remove impurities in the dry sweep air, such as dust or moisture, laboratory air from the fume hood was circulated through a drying unit (W. A. Hammond Drierite, Xenia, Ohio) and hollow Fiber Filter (Barnstead International, Beverly, Mass.) prior to flow into the permeate side. The drying unit helps to lower the relative humidity close to zero. In all experiments, the air flow rate was maintained at 4.5 L/min. The experiments were performed thrice and the relative standard deviation was observed to be below 1%.

The change in volume between the original feed and the recirculated feed was estimated to measure the permeate concentration. The reduction in feed volume was measured after 1 h of the experiment and the ethanol-water mixture composition before and after the experiment were evaluated using a UV spectrophotometer (UV-1800 UV-Vis Spectrophotometer, Shimadzu). At 190 nm, ethanol exhibited maximum absorbance (λₘₐₓ). A calibration curve was plotted for ethanol concentration vs absorbance at room temperature to measure the unknown concentration of ethanol after each experiment. The flux and separation factor for ABE-water mixture were calculated by analyzing the initial and final feed mixture compositions using gas chromatography (HP-5890) equipped with FID detectors. The gas chromatograph was operating with injection port temperature of 200° C., column temperature of 150° C., and detector temperature of 250° C. Analyses were carried out on an EzChrom Elite Chromatography data system used for GC control, data acquisition, and processing.

The PV tests were performed in a semi-continuous laboratory-scale setup. A 10:90 wt % water-ethanol feed solution (200 mL) was poured in the feed tank 62. The operating temperature (at 22, 40, 50° C.) was controlled using a thermometer, which was placed inside the membrane cell 64 (in contact with the azeotropic mixture). The vacuum on permeate side was set at 0.6 bar using a pressure gauge 66 (30 Hg/0 PSI).

The membranes, with an area of 12.5 cm², were located on a porous support within the membrane cell 64. The permeated vapor was condensed and collected in a glass trap placed in a liquid nitrogen condenser 68. After achieving the steady-state, the permeate was collected for 4 h and weighted to calculate the total permeate flux.

Of note, the weight percentages in the experimental work reported herein was 7.913 weight % of ethanol in feed and 92.087 weight % of water, and in the permeate, the weight percentages were 57.77 weight % of ethanol and 42.23 weight % of water, which is equivalent to 73 volume % of ethanol in the permeate.

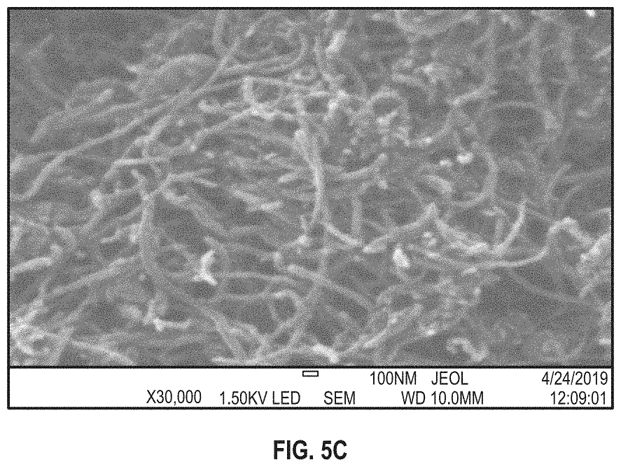

Now referring to FIGS. 5A-5C, SEM images of the surfaces of a plain PTFE substrate, CNIM, and CNIM-ODA are shown, respectively. The porous structure of the pristine PTFE membrane and presence of CNT and CNT-ODA on the CNIM and CNIM-ODA surfaces are clearly visible. Uniform distribution of CNTs over the entire membrane surface was also observed.

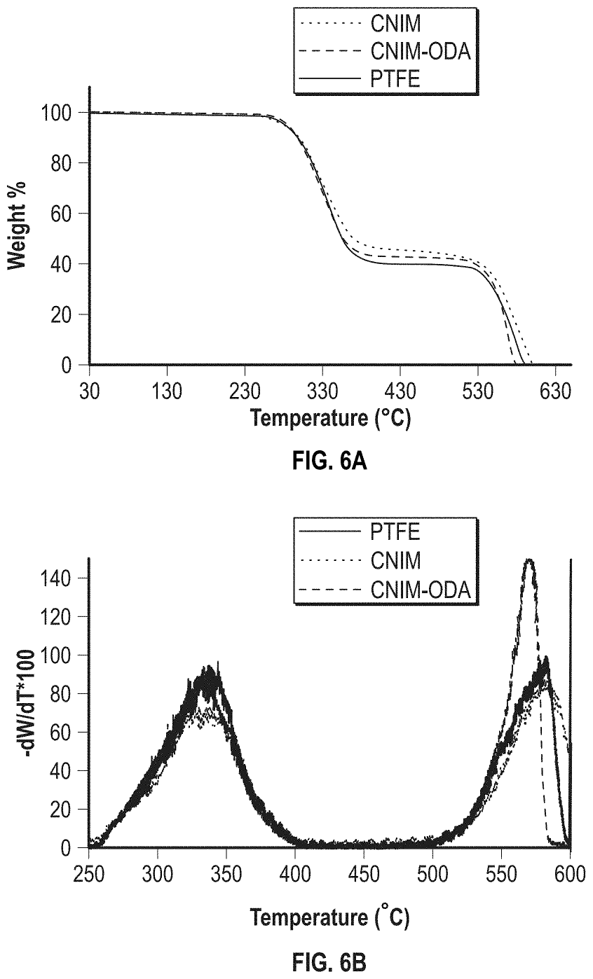

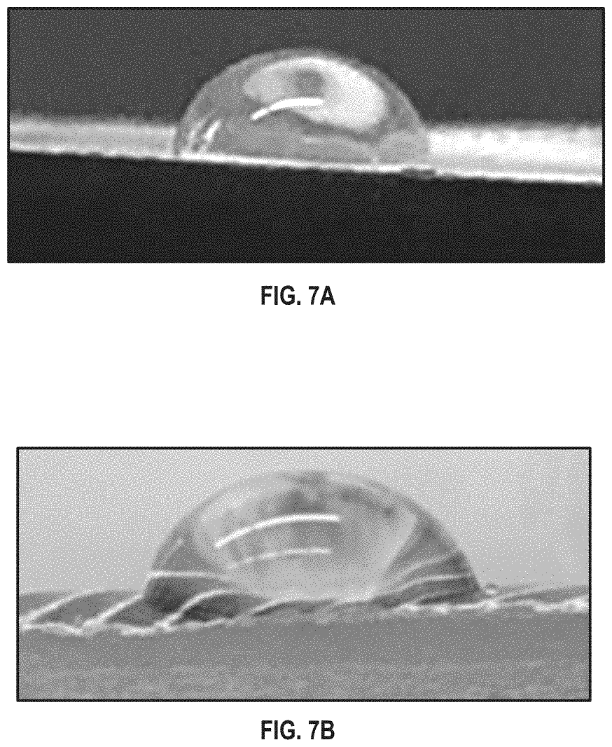

Turning now to FIGS. 6A and 6B, thermal degradation behavior and thermal stability of the PTFE, CNIM, and CNIM-ODA membranes was studied by thermogravimetric analysis (TGA). FIG. 6A reflects the TGA curve of the fabricated membranes. It is clear from FIG. 6A that the membrane is quite stable at moderate temperature. The TGA curve of the membrane showed its first weight loss stage occurring at 260° C. followed by a sharp decomposition at 500° C. indicating degradation of the PTFE layer. FIG. 6A also demonstrates a slight increase in thermal stability for the CNIM and CNIM-ODA due to the presence of CNTs in the polymer matrix. FIG. 6B shows the DSC curves of the fabricated membranes. A relatively high glass transition temperature was observed at 250° C.

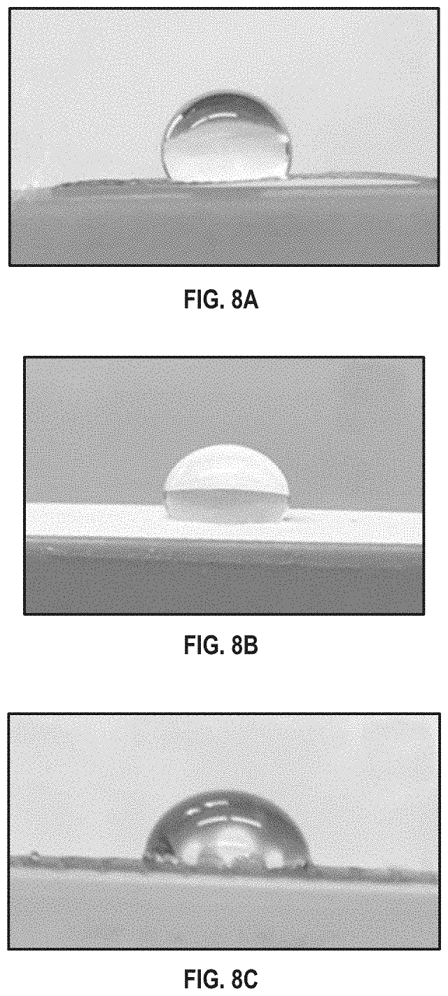

Referring to FIGS. 7A, 7B, & 8A-8C and Table 1, the contact angles for pure water were much higher on CNIM and CNIM-ODA due to their higher hydrophobicity, which were similar to what has been, reported previously [2-4].

TABLE 1
Contact Angles in degrees
with PTFE membrane and CNIM (°)

| Solvent | PTFE | CNIM |
|---|---|---|
| Pure water | 105 | 89 |
| 10% ethanol | 88 | 80 |
| 20% ethanol | 53 | 50 |

Referring to Table 2, the droplet of ABE-water mixture on CNIM indicated a contact angle of 84° vs a contact angle of 103° for PTFE and 108° indicating strong interactions with the CNTs and relatively less with CNT-ODA. The increasing ABE affinity to CNIM and CNIM-ODA over PTFE are potential means to increase the removal efficiency and reduce concentration polarization[5].

TABLE 2
Contact Angles of
pure water & ABE mixture

| | Contact angle (°) | | |
|---|---|---|---|
| Solvent | PTFE | CNIM | CNIM-ODA |
| Pure water | 105 | 109 | 116 |
| ABE mixture | 103 | 84 | 110 |

### Performance of CNIM, CNIM-ODA & PTFE

Ethanol-water separation and ABE-water separation were quantified based on flux and separation factor. The performance of CNIM, CNIM-ODA, and a commercial PTFE membrane were compared. The solvent vapor flux, J_{w}, across the membrane was defined as

$${J}_{w} = \frac{{W}_{p}}{t \times A} \qquad ( 1 )$$

where, Wp was the total mass of the permeate, t is the time and A is the effective membrane surface area.

Selectivity was quantified as a separation factor, which was a measure of preferential transport of an organic solvent and was defined as

$${α}_{solvent - water} = \frac{{y}_{solvent} / {y}_{water}}{{x}_{solvent} / {x}_{water}} \qquad ( 2 )$$

where yᵢ and xᵢ are the weight fraction of the component ‘i’ in permeate and feed, respectively.

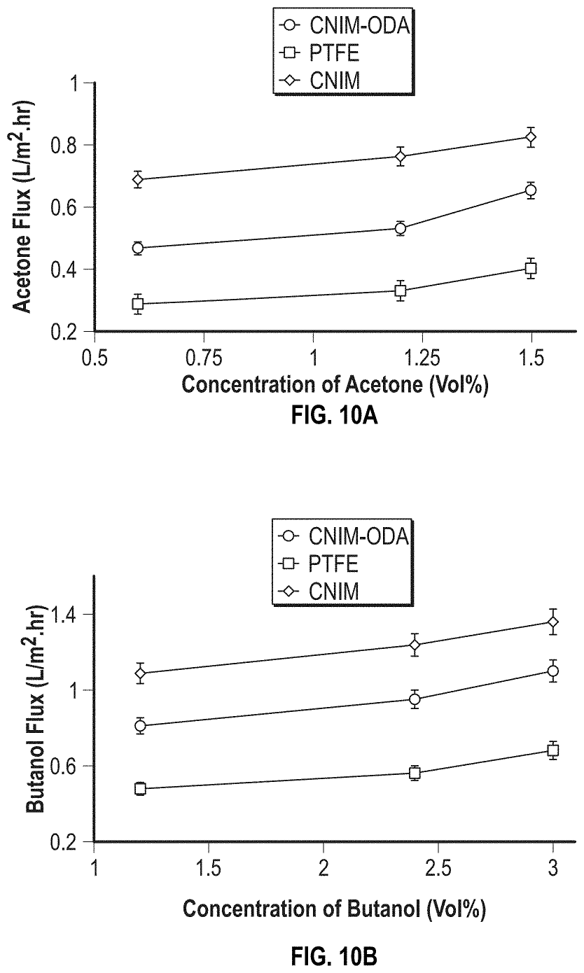

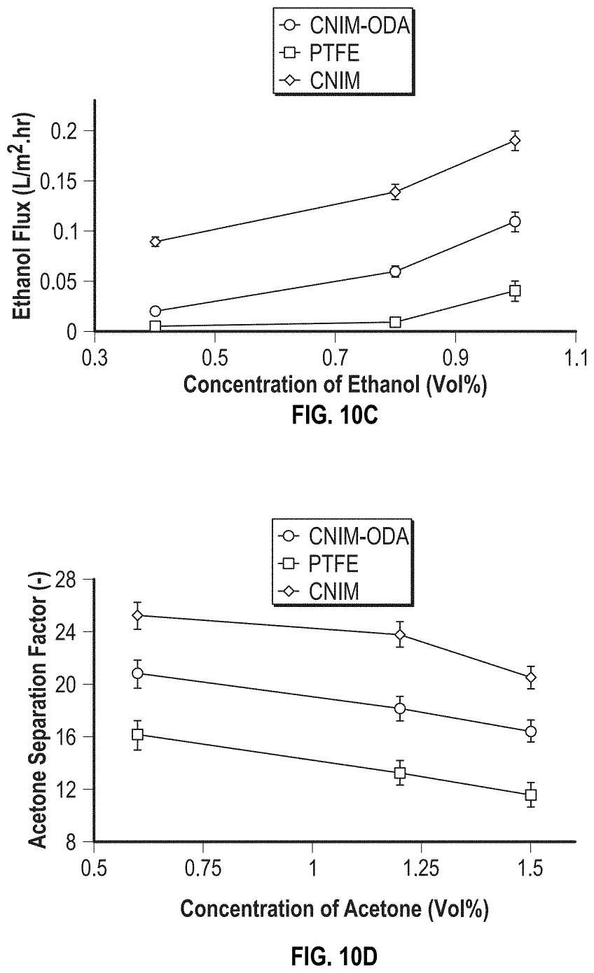

Now referring to FIG. 9A, the ethanol flux and separation factor is shown at various ethanol concentrations in the feed with CNIM and PTFE membranes, respectively. It is evident from FIG. 9A that for both membranes, the flux increased as the partial vapor pressure increases with an increase in ethanol concentration in the feed mixture. The flux as well as separation factors were significantly higher when CNIM was used. The CNTs enhanced the partition coefficient of ethanol on the membrane due to its higher affinity toward organic moiety and its influence was more evident when the ethanol concentration was high. However, both membranes showed a decline in ethanol separation factor with an increase in ethanol concentration. Ethanol flux reached as high as 11.3 L/m².hr with CNIM, which was 32% higher than unmodified PTFE membrane under the same conditions of 15 volume % of ethanol in feed at 50° C. The separation factors in CNIM were also significantly higher than unmodified PTFE membrane reaching an enhancement upto 28%.

Now referring to FIG. 9B, the influence of temperature on ethanol flux and separation factor with CNIM and PTFE membrane at a feed flow rate of 112 mL/min and sweep gas flow rate of 4.5 L/min is presented. The ethanol concentration in the feed was kept constant at 10% (v/v). It can be seen that the ethanol flux increased with increase in feed temperature, because at a higher feed temperature the driving force for solvent vapor transport increased due to higher vapor pressure difference across the membrane. Based on FIG. 9B, at 60° C., the ethanol vapor fluxes reached 10.6 L/m².h for CNIM, which was 40% higher than unmodified PTFE membrane.

Mechanistically speaking, the selective adsorption of the ethanol on the CNTs played a significant role in enhancing the performance dramatically. The enhancement in ethanol flux did not show any particular trend with increase in temperature, however, an increment in separation factor was observed for both membranes. In general, the flux enhancement with CNIM was anywhere between 40-48% and separation factor enhancements were between 40-80%.

Overall, CNIM displayed consistently improved ethanol vapor flux and better selectivity. The effects were more apparent at reduced feed temperatures. Therefore, the CNIM approach represents a major improvement in the state of the art.

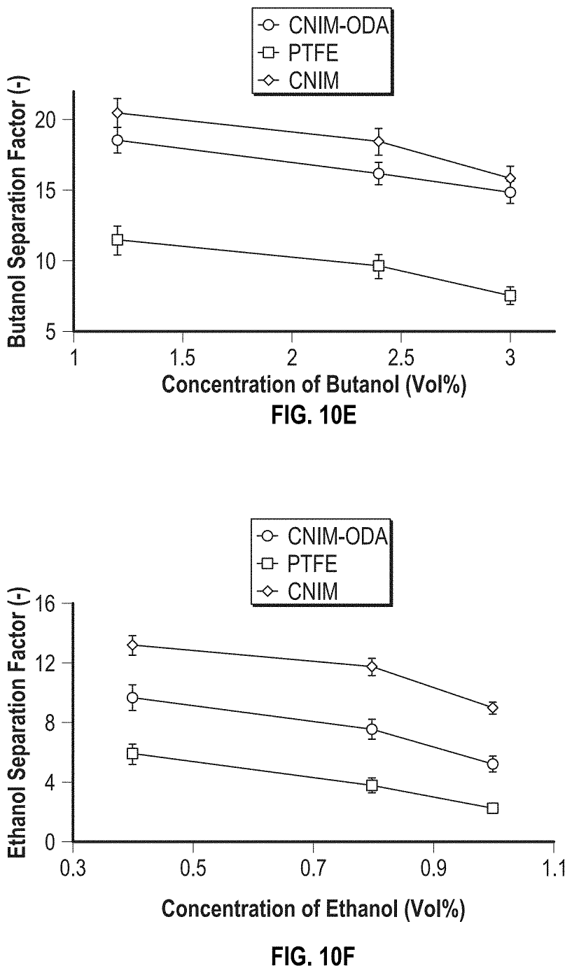

Now referring to FIG. 10A-FIG. 10C, the effect of feed concentration on acetone, butanol, and ethanol flux and separation factor is presented. Three different feed concentrations namely 0.6, 1.2, and 1.5 (vol %) of acetone were tested and the butanol and ethanol in the feed solutions were adjusted accordingly. The feed temperature and the feed flow rate were maintained at 40° C. and 112 mL/min, respectively. It can be observed from the figures that with increase in acetone, butanol, and ethanol concentration in feed, the ABE flux increased for all membranes. The CNIM and CNIM-ODA showed improved flux compared to the PTFE membrane, which was due to the enhanced solvent affinity with the nanotubes. Total solvent flux were in the order of CNIM>CNIM-ODA>PTFE. The highest total solvent flux for CNIM may be attributed to the higher solvent sorption capacity, as also supported by the contact angle values. The presence of bulky ODA groups on CNT-ODA may have limited the direct sorption and fast transport of the organic compounds on the CNT framework. The solvent flux reached as high as 0.82, 1.36 and 0.19 L/m².h for acetone, butanol, and ethanol, respectively, at 40° C. and 1.5, 3, and 1 vol % of ABE in the feed. The CNTs influenced the acetone, butanol, and ethanol partition coefficient, and its effects were more pronounced at higher concentrations. The enhancement in acetone flux reached as high as 130.3% for CNIM and 60.6% for CNIM-ODA over PTFE membrane at 1.2 volume % of acetone. Enhancement in butanol and ethanol flux followed similar pattern with enhancement reaching up to 127% and 375% respectively for CNIM.

Now referring to FIG. 10D-FIG. 10F, plots of separation factor of ABE with respect to feed concentration are shown. As can be seen from the plots, the separation factor was inversely proportional to the concentration for all the membranes. However, a higher separation factor for CNIM than CNIM-ODA and PTFE membrane was observed at all feed concentrations tested herein. Enhancement over PTFE membrane for acetone reached as high as 79.92% for CNIM and 41.5% for CNIM-ODA. Similar trends were observed for ethanol and butanol separation factor.

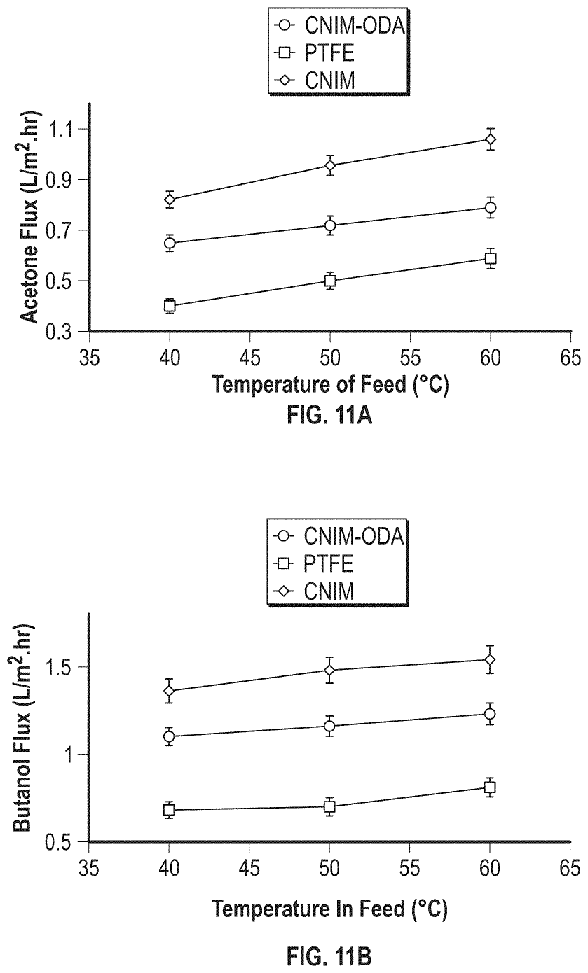

Now referring to FIG. 11A-FIG. 11F, the acetone, butanol and ethanol flux and separation factor on the CNIM, CNIM-ODA, and the PTFE membranes as a function of feed temperatures are demonstrated. A feed concentration of 1.5, 3, and 1 volume % of acetone, butanol, and ethanol, respectively, was maintained and the feed flow rate was kept constant at 112 mL/min. The permeate fluxes for all membranes showed a direct relationship with feed temperature. At 60° C., the CNIM flux reached up to 1.15 L/m².h, 1.54 L/m².h, and 0.58 L/m².h for acetone, butanol, and ethanol, respectively, which were considerably (around ten times) higher than previously reported data for pervaporation. In general, higher fluxes at all temperatures for CNIM were observed followed by CNIM-ODA, although the enhancement was distinct at reduced temperature. At 40° C., the improvement in acetone, butanol, and ethanol flux reached to 105, 100, and 375%, respectively, in comparison with pristine PTFE membrane. Hence, it is possible to perform the experiments at a relatively lower temperature thereby making it a less energy intensive process. It is well known that the vapor pressure increases exponentially with temperature and the sharp increase in vapor pressure from 40 to 60° C. was reflected in the corresponding increase in ABE flux. From FIG. 11D-FIG. 11F, it can be observed that at all the operating temperatures; CNIM's separation performance was significantly better compared to the commercial PTFE membrane. The separation factor enhancement of CNIM compared to PTFE membrane reached to 103, 129, and 324% at 50° C. for ABE. As a result of negative effects of viscosity, a decline in ABE separation factor was observed with increase in operating temperatures for all membranes [6].

It was important to investigate if separation of each ABE component was affected by the presence of the others. Therefore, binary mixture of each compound with water was also studied using PTFE and CNIM. Of note, with increase in feed concentration, the flux increases for each compound in both membranes. Butanol, which had limited miscibility with water, has shown higher flux than ethanol that was significantly more miscible. As expected, higher flux was obtained for all solvents when CNIM was used. It has been observed that the individual solvent flux in the binary mixtures was higher compared to the ABE mixture under similar condition. For example, the acetone flux was obtained to be 1.36 L/m² hr for CNIM at 40° C. and 1.5 vol % of acetone in water, which was 65.8% higher than the corresponding ABE mixture. Similar trend was also observed for butanol and ethanol mixture. The flux decline in the case of a mixture may be attributed to the mutual interaction and competition between the different compounds that reduced portioning as well as permeability[7].

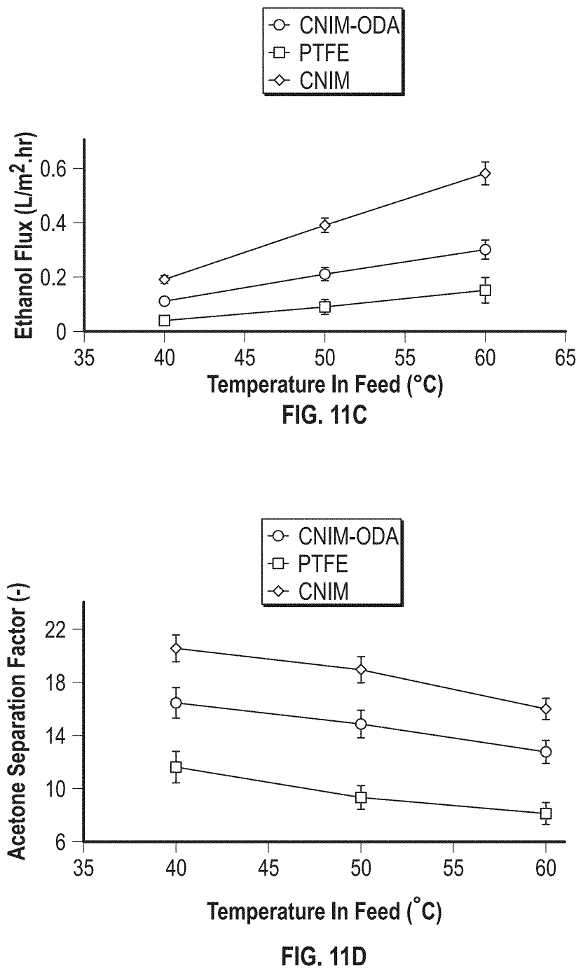

Now referring to FIG. 12, the effect of feed water concentration and alcohol content on the total permeate flux during the PV performance is displayed. Essentially, an increment in the total permeation rate was observed with a GO-CNT composite membrane. This tendency is commonly observed during the incorporation of the inorganic materials into polymer membranes, which may be a result of the free volume increase as well as the possible interfacial selective gaps between GO sheets and PVA matrix, while the highly hydrophilic nature of the filler can also produce a raise in the permeation rates by preferential adsorption of the more polar compound (water). In theory, the polymer chains tend to be more flexible at higher temperatures promoting the sorption ability of the components, leading to the increase of permeating compounds through the intermolecular distances of the polymeric membrane. A decrease in separation factor is observed as a function of the concentration of alcohol for pure PVA membrane as well as its MMMs. Certainly, the decrease of separation factor in the MMMs might be due to the combined effect of several factors, such as characteristics of GO and CNT (e.g., the structure and the influence of its preparation procedure), polymer properties, the effect of the cross-linking procedure on the adsorption capacity of the polymer, and the operating temperature. Herein, the simultaneously increased permeation flux and separation factor suggested that the incorporation of CNTs and GO could: tune the packing of hydrophilic chains in the interface, thereby generating increased free volume and appropriate free volume cavity size; additionally provide internal nanochannels for water permeation from the nanoscale opening of CNTs.

The mass transfer coefficient k was calculated from flux J_{w} as:

J_{w}=k(P_{f}−Pₚ) (3)

where, P_{f} and P_{P} are the partial pressure in feed and permeate side. Usually, Pₚ is considered as zero, since dry air was used as sweep gas.

Table 3 presents the variation in mass transfer coefficient in PTFE & CNIM at different feed temperatures.

TABLE 3
Mass transfer coefficients with conventional
and microwave heating for PTFE and CNIM

| | Mass Transfer Coefficient (MIMD) | |
|---|---|---|
| Temperature | (m/s mm Hg) | |
| (° C.) | PTFE | CNIM |
| 40 | 2.58E−1 | 3.81E−1 |
| 50 | 2.02E−1 | 2.71E−1 |
| 60 | 1.61E−1 | 2.05E−1 |

It can be seen from Table 3 that the mass transfer coefficients decreased at higher temperatures. This can be attributed to the fact that the effect of concentration and temperature polarization increase at higher temperatures. The CNTs are known to provide rapid sorption/desorption properties, which contributed to high mass transfer coefficients. The enhancement of mass transfer coefficient reached as high as 48% at 40° C. The overall mass transfer coefficient depended upon the partitioning of the ethanol on the CNIM surface as well as the diffusion through the membrane. While the former decreased with temperature, the latter increased with temperature. In this case, the overall coefficient was the highest at 40° C. This may be due to the decrease in sorption capacity at higher temperatures, which led to a reduction in enhancement of separation factor at higher temperatures.

The ‘kᵢ’ values of different components in ABE mixture at varied operating temperatures and a constant feed flowrate of 112 mL/min are presented in Table 4.

TABLE 4
Mass transfer coefficient of ABE at different temperature
and 1.5, 3 & 1 vol % ABE feed at 112 mL/min.

| | Mass transfer coefficient (×10⁻³ L/m² · h · mm-Hg) | | | | | | | | |
|---|---|---|---|---|---|---|---|---|---|
| Temp | PTFE | | | CNIM | | | CNIM-ODA | | |
| (° C.) | Acetone | Butanol | Ethanol | Acetone | Butanol | Ethanol | Acetone | Butanol | Ethanol |
| 40 | 0.95 | 35.9 | 0.30 | 1.94 | 71.9 | 1.42 | 1.54 | 58.1 | 0.82 |
| 50 | 0.97 | 20.2 | 0.41 | 1.57 | 42.7 | 1.77 | 1.18 | 33.5 | 0.95 |
| 60 | 0.68 | 13.4 | 0.43 | 1.23 | 25.4 | 1.65 | 0.73 | 20.3 | 0.86 |

The mass transfer coefficients decreased or remained almost constant with increase in operating temperature for CNIM, CNIM-ODA, and PTFE membranes. At all feed temperatures, the CNIM exhibited higher ‘kᵢ’ than the pristine PTFE membrane and CNIM-ODA. The enhancement of mass transfer coefficient over PTFE reached as high as 105% for CNIM and 62.5% for CNIM-ODA for acetone, 100% and 61.8% for butanol, and 375% & 175% for ethanol at 40° C. For butanol, the mass transfer coefficient follows an inverse relationship with temperature for all membranes. As mentioned earlier, at higher temperatures the temperature polarization increases significantly, resulting in a lower membrane mass transfer coefficient [8].

### Membrane Stability

To explore the stability of the membranes in presence of these strong organic solvents, SGMD experiments were performed for 8 h a day for 60 days with 1.5, 3, and 1 vol % of ABE concentration, respectively. The temperature was maintained at 60° C. The ABE flux was measured periodically. No substantial alteration in flux and membrane wetting were detected even during extended use for all membranes. It can be assumed that there was no significant CNTs loss from the membrane surface as it was not detected in the recycled feed solutions. Comparable stability checks in the past had been implemented where CNIM was used in high temperature aqueous solutions for extended periods and then examined for CNT loss [9].

### Proposed Mechanism

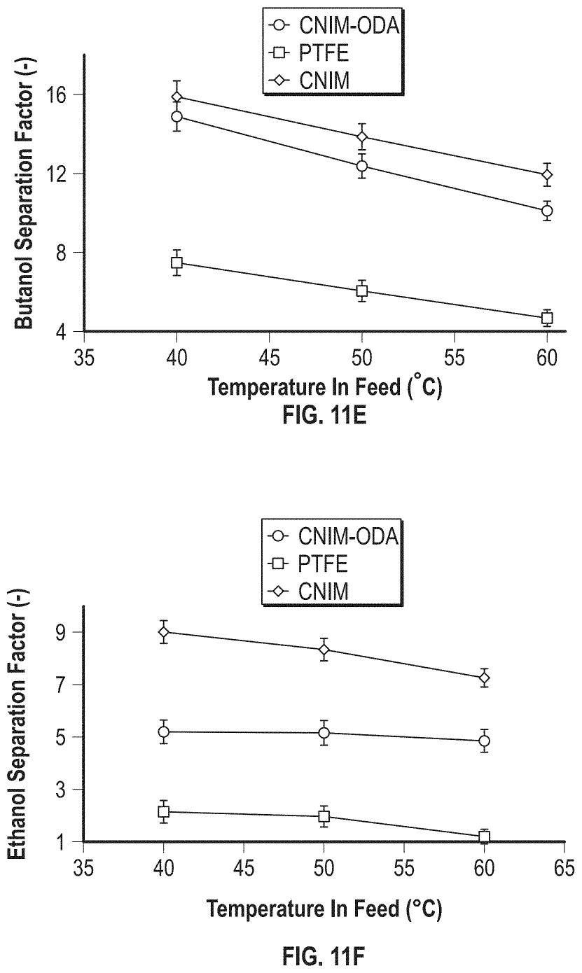

With reference to FIG. 13, during conventional heating, the entire volume of the feed stream is uniformly heated. On the contrary, microwave heating involves direct heating of the feed mixtures resulting in localized superheating. The dielectric loss of ethanol is known to increase with temperature, whereas for water, it decreases with temperature. Therefore, the microwave dissipation can be more significant in hot areas and can lead to local turbulence and spatial temperature gradients [10, 11]. The localized super heating and breaking of hydrogen bonded ethanol-water clusters are bound to enhance the tendency of ethanol molecules to escape from the feed mixture resulting in improved flux and better separation efficiency.

With further reference to FIG. 13, previous studies with CNTs have demonstrated that CNTs are excellent sorbents that enhance partition coefficient of the solutes leading to higher flux in membranes. The significant enhancement in ethanol flux with CNIM is attributed to the preferential sorption and fast desorption to the permeate side via CNTs serving as nanosorbents. The organophilic CNT surface is selective toward ethanol due to its organic nature. Ethanol also has a higher vapor pressure of 348.83 mm Hg at 60° C., which is almost double of that of water (149.038 mm Hg at the same temperature). In summary, the higher vapor pressure of ethanol, preferential sorption, and permeation of the ethanol via CNTs offer higher enhancement in flux and separation factor.

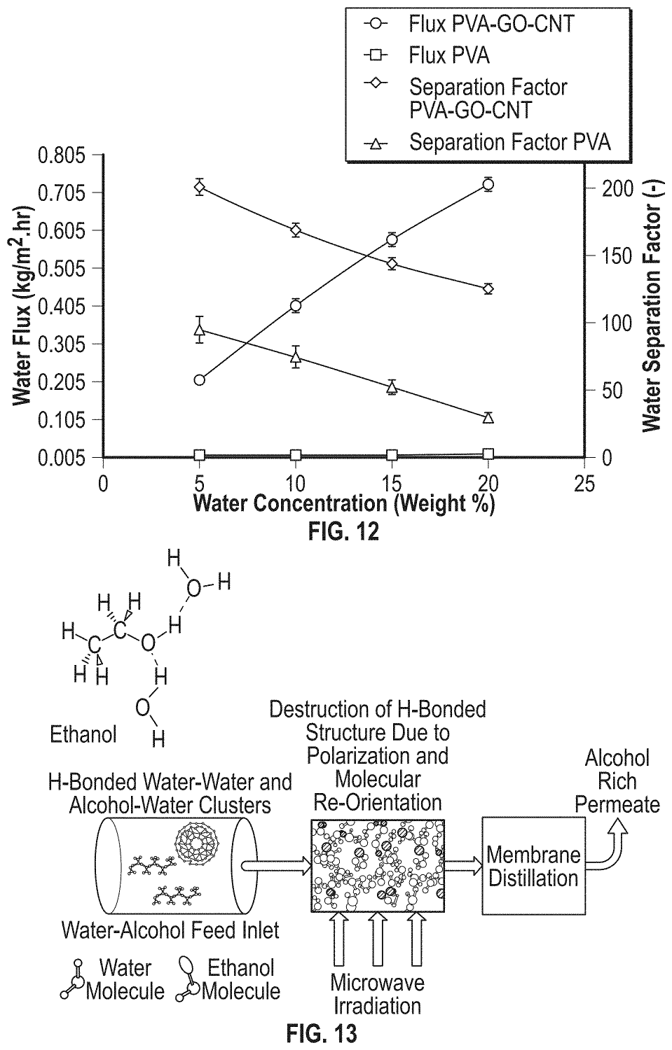

Now referring to FIG. 14, the enhanced ABE transport mechanism with CNIM is presented. Earlier research published with CNTs has validated that CNTs are exceptional sorbents that increase solute partition coefficient generating higher permeation rate through the membranes. The CNTs are also known to facilitate fast mass transport in both separation processes including chromatography, sorbents, and membranes. The higher vapor pressure of acetone, butanol, and ethanol compared to water helped in selective sorption and penetration of ABE mixture through the porous membrane at low temperature. The significant enhancement in ABE flux and separation factors in CNIM and CNIM-ODA are attributed to these multiple factors.

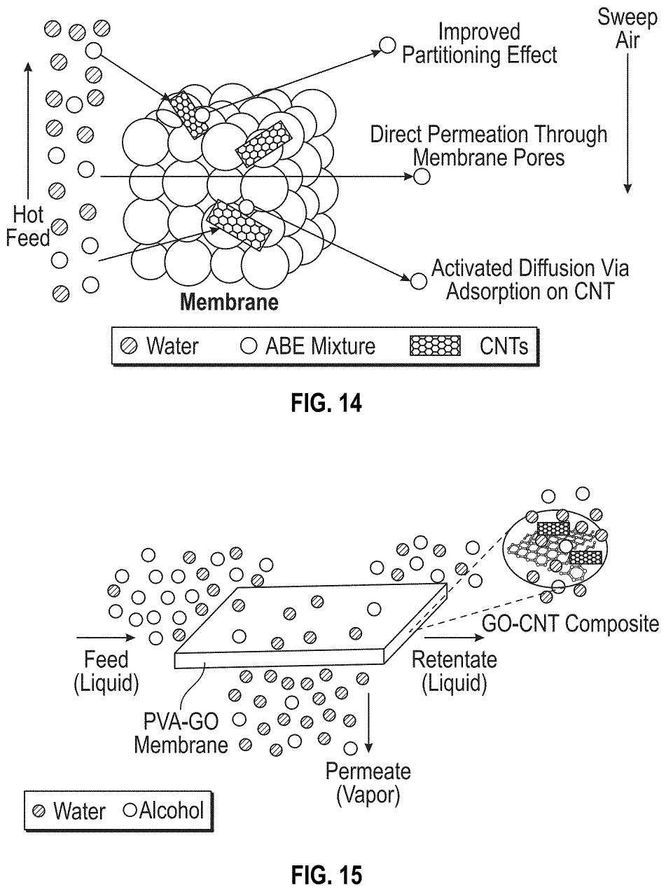

With reference to FIG. 15, pervaporation for removal of small quantities of volatile organic compounds from water using hydrophobic membranes suffers from limitations such as low flux. Thus, combining MD with pervaporation is advantageous where concentrating the alcohol to remove excess water can be industrially significant for its use as a solvent in pure form such as in manufacturing of cosmetics and as a cleaning agent in semiconductor industries. Pure ethanol can also be blended with gasoline to form biofuels to reduce environmental impact from greenhouse gases and develop a sustainable environment. A hybrid process exploits the advantages of both techniques, while minimizing the negative impacts. For example, negative impacts of the PV process such as membrane stability, concentration and temperature polarization, and the temperature drop that occurs in the liquid are factors that increase the required membrane area, the amount of secondary equipment, and the capital and operating cost. Hybrid processes are also very promising especially in cases where high product purities are required. CNT-GO showed preferential affinity toward water; both CNT and GO could modify PVA polymer chain packing, which further increased free volume and tailored free volume cavity size of PVA membrane.

Although the systems and methods of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refine ments are encompassed within the scope of the present invention. All references listed and/or referred to herein are incorporated by reference in their entireties.

### REFERENCES

[1] S. Roy, R. S. Petrova, S. Mitra, Effect of carbon nanotube (CNT) functionalization in epoxy-CNT composites, Nanotechnology Reviews, 7 (2018) 475-485.
[2] O. Gupta, S. Roy, S. Mitra, Enhanced membrane distillation of organic solvents from their aqueous mixtures using a carbon nanotube immobilized membrane, Journal of Membrane Science, 568 (2018) 134-140.
[3] M. Bhadra, S. Roy, S. Mitra, Flux enhancement in direct contact membrane distillation by implementing carbon nanotube immobilized PTFE membrane, Separation and Purification Technology, 161 (2016) 136-143.
[4] M. Bhadra, S. Roy, S. Mitra, A bilayered structure comprised of functionalized carbon nanotubes for desalination by membrane distillation, ACS applied materials & interfaces, 8 (2016) 19507-19513.
[5] S. O. Olatunji, L. M. Camacho, Heat and Mass Transfer in Modeling Membrane Distillation Configurations: A Review, Frontiers in Energy Research, 6 (2018) 130.
[6] C. H. Lee, W. H. Hong, Effect of operating variables on the flux and selectivity in sweep gas membrane distillation for dilute aqueous isopropanol, Journal of Membrane Science, 188 (2001) 79-86.
[7] H. Zhou, Y. Su, X. Chen, Y. Wan, Separation of acetone, butanol and ethanol (ABE) from dilute aqueous solutions by silicalite-1/PDMS hybrid pervaporation membranes, Separation and Purification Technology, 79 (2011) 375-384.
[8] J. Phattaranawik, R. Jiraratananon, Direct contact membrane distillation: effect of mass transfer on heat transfer, Journal of Membrane Science, 188 (2001) 137-143.
[9] S. Roy, M. Bhadra, S. Mitra, Enhanced desalination via functionalized carbon nanotube immobilized membrane in direct contact membrane distillation, Separation and Purification Technology, 136 (2014) 58-65.
[10] R. Brand, P. Lunkenheimer, U. Schneider, A. Loidl, Excess wing in the dielectric loss of glass-forming ethanol: a relaxation process, Physical Review B, 62 (2000) 8878.
[11] W. Routray, V. Orsat, Dielectric properties of concentration-dependent ethanol+ acids solutions at different temperatures, Journal of Chemical & Engineering Data, 58 (2013) 1650-1661.

1. A membrane distillation and pervaporation system, comprising (i) a membrane distillation system comprising a sweep gap membrane distillation module and a microwave unit, and (ii) a pervaporation system in fluid communication with the membrane distillation system, the pervaporation system comprising a membrane module and a liquid nitrogen trap.

2. The membrane distillation and pervaporation system of claim 1, wherein the microwave unit is configured to effect microwave irradiation of a feed solution.

3. The membrane distillation and pervaporation system of claim 1, wherein the liquid nitrogen trap is adapted to condense a permeated component.

4. The membrane distillation and pervaporation system of claim 1, wherein the membrane distillation system further comprises a feed inlet to receive an aqueous feed solution and a feed outlet, a condensing medium inlet and outlet to obtain a condensing medium and to remove a stream of solvent vapor from the sweep gap membrane distillation module.

5. The membrane distillation and pervaporation system of claim 4, wherein the condensing medium is a sweep gas.

6. The membrane distillation and pervaporation system of claim 1, further comprising a flowmeter to measure a feed flow rate.

7. The membrane distillation and pervaporation system of claim 1, wherein the membrane distillation system includes a carbon nanotube immobilized on a polytetrafluorethylene surface.

8. The membrane distillation and pervaporation system of claim 7, wherein the carbon nanotube immobilized on a polytetrafluorethylene surface is disposed on a porous substrate.

9. The membrane distillation and pervaporation system of claim 1, wherein the pervaporation system further comprises a feed inlet to receive an alcohol solution containing a trace amount of water and a feed outlet, a condensing medium inlet and outlet to obtain a condensing permeate and to recirculate a stream of solvent from the membrane module.

10. The membrane distillation and pervaporation system of claim 9, wherein the condensing medium comprises communication with a vacuum source.

11. The membrane distillation and pervaporation system of claim 1, wherein the membrane module includes a polymeric dense membrane which includes a polyvinyl alcohol base and graphene oxide to form a mixed matrix membrane.

12. The membrane distillation and pervaporation system of claim 1, wherein the membrane module is selected from the group consisting of a hollow fiber membrane module, a flat membrane module, and a spiral wound membrane module.

13. A membrane distillation and pervaporation system, comprising:
a. a membrane distillation system comprising a carbon nanotube immobilized membrane, and
b. a pervaporation system in fluid communication with the membrane distillation system, the pervaporation system comprising a dense membrane.

14. A method for downstream recovery of a fermentation product, the method comprising the steps of:
a. separating alcohol from a water alcohol mixture upon introduction of the water alcohol mixture to a carbon nanotube immobilized membrane to recover alcohol with trace amounts of water, and
b. further separating the alcohol from the alcohol with trace amounts of water upon introduction of the alcohol with trace amounts of water to a dense membrane to recover substantially pure alcohol.
